(19) United States
(12) Patent Application Publication
(10) Pub. No.: US 2016/0030722 A1
(43) Pub. Date: Feb. 4, 2016

(54) METHOD AND APPARATUS FOR WOUND DRESSING

(71) Applicant: THE GENERAL HOSPITAL CORPORATION, Boston, MA (US)

(72) Inventors: RICHARD R. ANDERSON, Boston, MA (US); MARTIN PURSCHKE, Dorchester, MA (US); WALFRE FRANCO, Westborough, MA (US); JOSHUA TAM, Charlestown, MA (US); YING WANG, Malden, MA (US); WILLIAM FARINELLI, Danvers, MA (US)

(21) Appl. No.: 14/777,097

(22) PCT Filed: Mar. 14, 2014
(86) PCT No.: PCT/US2014/029306

§ 371 Date: Sep. 15, 2015

Related U.S. Application Data
(60) Provisional application No. 61/798,849, filed on Mar. 15, 2013.

(51) Int. Cl. A61M 35/00; A61M 1/00; A61F 13/02

(52) U.S. Cl. CPC A61M 35/00; A61F 13/0216; A61F 13/023; A61M 1/0088; A61M 1/0023; A61M 2202/0208; A61M 2205/75; A61M 2205/051

(57) ABSTRACT

Exemplary methods and devices can be provided for an improved wound dressing that facilitates healing. For example, the dressing can include a membrane that maintains a sterile enclosed volume over the wound. Pressure in the enclosed volume can be reduced by deforming the membrane and compressing a resilient open-cell sponge provided therein, facilitating a relatively unobstructed flow of air out of the enclosed volume. Oxygen and/or moisture can be introduced by a controlled flow of moist oxygen-containing gas into the enclosed volume. An oxygen-producing reaction within the enclosed volume using calcium peroxide or the like can also provide oxygen to the wound site. An external vacuum source that includes compressible foam can also be coupled to the enclosed volume to provide a reduced pressure therein. The external vacuum source can be attached to a user's body to maintain the reduced pressure without use of electricity.

### CROSS-REFERENCE TO RELATED APPLICATION(S)

The Present application relates to and claims priority from U.S. Provisional Patent Application Ser. No. 61/798,849 filed Mar. 15, 2013, the present disclosure of which is incorporated herein by reference in its entirety.

### FIELD OF THE PRESENT DISCLOSURE

The present disclosure is directed to wound healing, and more specifically to a method and apparatus for protecting a wound in biological tissue to improve the healing process.

### BACKGROUND INFORMATION

Many types of large open wounds, such, as those associated with tissue grafts, burns, abrasions, ulcers and the like, require careful treatment to facilitate their closure and healing. A number of factors have been identified that promote healing of such wounds. These factors include, e.g., a sterile environment to avoid infection, an adequate blood supply to the wound region, sufficient oxygen and hydration, a slight vacuum or negative pressure over the wound, mechanical force applied onto the wound to avoid pooling of liquids, proper drainage, etc.

Many types of wound dressings have been developed and studied. Maintaining a sterile environment around a wound can be achieved, for example, by sealing the wound within a closed environment. However, such a simple sealed dressing can also prevent oxygen from reaching the wound site, and may also lead to local accumulation of fluids that can inhibit healing. Ischemia, or lack of blood flow, can also inhibit healing of the wound. It has been observed that providing a reduced pressure or vacuum over the wound site can improve the healing process. However, existing systems that can provide such a reduced-pressure wound environment tend to be bulky, expensive and inconvenient, require a large power supply or connection to an external power source, etc., which can inhibit patient mobility. Conventional wound dressings and dressing systems typically represent compromises in which one or more of the significant factors mentioned herein that facilitate wound healing may be absent or inadequately provided.

Accordingly, it can be desirable to provide an improved wound dressing system and method that provides a plurality of conditions conducive to wound healing, while also allowing patient mobility and being relatively simple and cost-effective.

### SUMMARY OF THE PRESENT DISCLOSURE

Exemplary embodiments of the present disclosure provide method and apparatus for facilitating healing of wounds that address many factors beneficial to the healing process, including maintaining sterility, oxygenation and hydration, providing a reduced-pressure environment and a mechanical force on the wound site, and optionally other additional factors. Exemplary embodiments of the present disclosure can provide a wound dressing system that achieves these factors and can also be inexpensive, passive (e.g., it does not require electricity), and lightweight such that it may promote general mobility of the patient during the healing period.

In one exemplary embodiment of the present disclosure, system or apparatus can be provided that maintains an enclosed sterile environment around the wound under a reduced pressure (e.g. pressure that is below atmospheric pressure). The exemplary apparatus includes a gas-impermeable membrane that can be formed at least partially of a material such as Tegaderm® or the like, and sized to be placed over the wound and adhered to healthy tissue surrounding it. The apparatus can further include a resilient sponge e.g., a porous open-celled foam or similar material, enclosed by the membrane over the wound. A pore size of the sponge can be between, e.g., about 100 μm and about 1000 μm.

The sponge can optionally be infused with any of a variety of nutrients or other healing-promoting substances, and the lower surface of the sponge can be provided with a material or layer to reduce or prevent adhesion of the sponge to the wound, such as a woven Teflon® mesh or the like. The sponge can also facilitate removal of fluids exuded by the wound away from the wound site as it heals.

The membrane can include at least one inlet and at least one outlet provided therethrough, where the inlet can include a valve arrangement, such as an adjustable one-way valve, to facilitate a controlled flow of gases into the reduced-pressure environment at a controlled or selected rate, and the outlet can include a one-way valve that allows gases to exit the enclosed volume beneath the membrane while preventing intake of gases or liquids into this enclosed volume through the outlet. For example, an application of an external force to the membrane can deform it, and compress the sponge therein, forcing some gas to be expelled from the enclosed volume over the wound and through the outlet, and relaxation of the compressed sponge to an expanded state can then exert an outward force on the membrane, reducing pressure within the enclosed volume, while also providing a mechanical force upon the wound.

In further exemplary embodiments of the present disclosure, the one or more inlets can, include a valve arrangement structured to allow a controlled flow of a gas or liquid into the enclosed volume. For example, such gas flow, e.g., between about 0.1 ml/min and about 10 ml/min into the enclosed volume, can provide oxygen to the wound to promote healing. The gas can optionally be filtered prior to entering the enclosed volume by a filter arrangement. In certain embodiments, the incoming gas can also be moisturized by allowing the gas to pass through a wetted material, to hydrate the wound.

In further embodiments, the apparatus may be provided without a valved inlet through the membrane, and the membrane (or a portion thereof) can be gas-permeable to facilitate a flow of air or another gas through the membrane when a low pressure is provided in the enclosed. Such a permeable membrane can also filter the entering gas, which may be ambient air.

In still further exemplary embodiments of the present disclosure, the inlet can be provided with, a coupler that may be configured to attach to a tube, container, reservoir, or the like, to introduce liquid and/or gaseous substances through the inlet and into the enclosed volume over the wound. Such substances can include, e.g., oxygen, tissue-growth promoters, antibacterial compounds, or the like.

In another exemplary embodiment, a plurality of inlets can be provided at various locations through the membrane, e.g., to provide a more spatially uniform influx of gases and/or liquids into the enclosed volume.

In a further exemplary embodiment of the present disclosure, an external vacuum arrangement can be connected to the outlet of the membrane via a tube. The external vacuum arrangement can include a gas-impermeable housing that is at least partially deformable. A foam or spring-like structure can be provided in the interior of the housing or formed as a part thereof to generate a restorative force to the housing when it is compressed or deformed. The external vacuum arrangement can include one or more inlet ports that allow gases and/or liquids to flow from the enclosed volume to the interior volume of the housing, and prevent gases or liquids from leaving the interior of the housing through the inlets. A fluid path can be provided between the interior of the housing and the enclosed volume over the wound, e.g., via a tube or conduit. One or more outlets can be provided that allow gases to exit the housing when it is deformed but prevent gases from re-entering it through the outlets. The external vacuum arrangement can thereby provide a source of low pressure in the enclosed volume between the membrane and the wound when the housing is compressed to expel gas contained therein, and then withdraws gas from the enclosed volume and into the inlet(s) s the housing tries to expand back to a relaxed state.

A trap can optionally be provided within or proximal to the inlet of the housing, or it can be provided as part of or coupled to a tube connecting the external vacuum source to the enclosed volume under the membrane. Such trap can retain moisture, liquids, particles, impurities, or the like that may flow through or past the trap. The exemplary trap can be formed from one or more materials such as, e.g., a paper filter element, a woven material, a filter screen, an open-cell sponge or scaffold material, an absorbent material, or a combination of such materials.

In yet further embodiments of the present disclosure, an absorbent material can be provided proximal to the wound, e.g., near the periphery of the membrane 120. This absorbent material can absorb fluids produced at the wound site, e.g., to facilitate drainage of the wound as it heals.

A method for dressing a wound can be provided that includes, e.g., adhering a membrane to healthy tissue surrounding the wound to form an enclosed volume over the wound, providing oxygen and moisture to the wound, providing a reduced pressure environment for the wound, and providing a mechanical force on the wound. The method can further comprise providing oxygen and/or moisture to the wound site in a gas that is directed to flow into the enclosed volume through an opening provided in the membrane.

The reduced pressure can be provided by compressing the membrane to force air to flow out of the enclosed volume through a further opening provided in the membrane, where a sponge can be provided in the enclosed volume to provide a restorative force to the compressed or deformed membrane. If gas flow into the enclosed volume is restricted or prevented, a reduced pressure will be present in the enclosed volume as the membrane tries to expand.

In another exemplary embodiment of the present disclosure, the reduced pressure in the enclosed volume can be provided by compressing an external resilient housing to direct air to flow out of the enclosed volume through a second opening provided in the membrane and into the housing via a tube connecting the second opening and a further opening in the housing.

In further exemplary embodiments of the present disclosure, an external vacuum arrangement can be provided that is shaped and configured to be attached to a user's body part, placed in a clothing pocket, etc., and connected to the membrane over the wound by a tube. This vacuum arrangement can be configured to periodically undergo compression during normal bodily activity to maintain a reduced pressure in the enclosed volume over the wound without electricity, directed actions, or the like. In certain embodiments, the vacuum arrangement can be provided on the chest or stomach area, under an arm, behind a knee, etc., and can be affixed to the body using a strap or band, an adhesive, hook-and-loop closures, or the like. Such a configurations allow the vacuum arrangement to be activated by normal body motion such as breathing, arm movement, walking, etc. to maintain a reduced pressure in the enclosed volume over the wound.

In still further exemplary embodiments, one or more sensors can be provided with the wound dressing apparatus, such as, e.g., a pressure sensor, a pH sensor, an oxygen sensor, a moisture sensor, or the like. Such sensors can provide signals relating to the conditions of the wound site and may be used, for example, with a control arrangement to automatically open a valve arrangement to provide moisture or oxygen, adjust a temperature if a heater is provided, etc. Such sensors can also provide a notification of conditions at the wound site, for example, to indicate when maintenance of the wound site or dressing may be needed, e.g., to open a valve slightly to introduce more oxygen or moisture, etc. Such sensors can facilitate a maintenance of desirable conditions at the wound site.

In a further exemplary embodiment of the present disclosure, oxygen can be provided to the wound site via an oxygen-producing reaction within the enclosed volume. For example, calcium peroxide (CaO₂) can be provided within the enclosed volume, e.g., on a portion of the membrane or sponge, or on a separate object provided within the enclosed volume. CaO₂ can produce oxygen when contacted by water to form calcium hydroxide and oxygen. Other biocompatible reactions known in the art that produce oxygen can be used in a similar manner with the wound dressing system in further embodiments of the present disclosure.

In still further exemplary embodiments, at least a portion of the membrane and/or sponge can be formed of materials that transmit light having certain wavelengths, e.g., to facilitate irradiation of the wound from outside of the membrane. Such low-level irradiation using certain wavelengths can improve, enhance, or speed up the healing process. In further embodiments, one or more LEDs or other conventional light-emitting arrangements can also be provided on or affixed to the membrane. Such light-emitting arrangements can be battery-powered for portability, and can be configured to emit light at one or more wavelengths that enhance the healing process.

These and other objects, features and advantages of the present disclosure will become apparent upon reading the following detailed description of embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying figures showing illustrative embodiments, results and/or features of the exemplary embodiments of the present disclosure, in which:

FIG. 1A is a schematic cross-sectional illustration of a first wound dressing system in accordance with exemplary embodiments of the present disclosure;

FIG. 1B is a schematic cross-sectional side view of an exemplary sponge structure that can be used with the wound dressing system shown in FIG. 1A;

FIG. 1C is a schematic cross-sectional side view of an exemplary outlet configuration that can be used with the wound dressing system shown in FIG. 1A;

FIG. 1D is a schematic cross-sectional side view of another exemplary outlet configuration that can be used with the wound dressing system shown in FIG. 1A;

FIG. 2 is a schematic illustration of a second wound dressing system in accordance with further exemplary embodiments of the present disclosure; and

FIG. 3 is an exemplary vacuum arrangement that can be used with certain exemplary embodiments of the present disclosure.

Throughout the drawings, the same reference numerals and characters, unless otherwise stated, are used to denote like features, elements, components, or portions of the illustrated embodiments. Similar features may thus be described by the same reference numerals, which indicate to the skilled reader that exchanges of features between different embodiments can be done unless otherwise explicitly stated. Moreover, while the present disclosure will now be described in detail with reference to the figures, it is done so in connection with the illustrative embodiments and is not limited by the particular embodiments illustrated in the figures. It is intended that changes and modifications can be made to the described embodiments without departing from the true scope and spirit of the present disclosure as defined by the appended claims.

### DETAILED DESCRIPTION OF EXEMPLARY EMBODIMENTS

The present disclosure relates to various exemplary embodiments of methods and apparatus for dressing a wound by providing a sealed sterile environment around the wound under a reduced pressure (e.g. pressure that is below atmospheric pressure). Exemplary embodiments of the present disclosure can also facilitate healing by providing oxygen and/or certain nutrients to the wound site, and can maintain a mechanical force on the wound to further enhance the healing process.

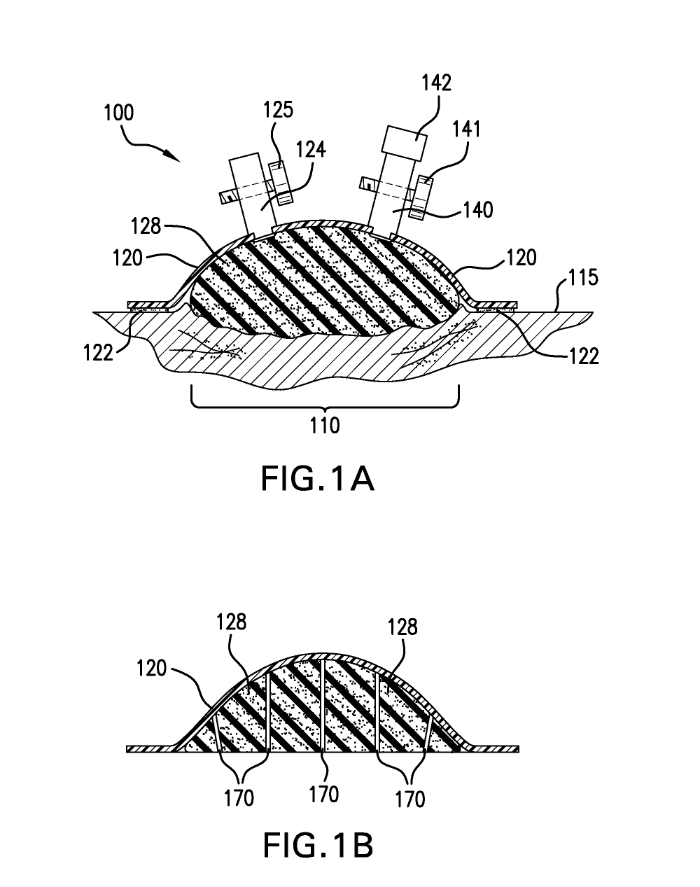

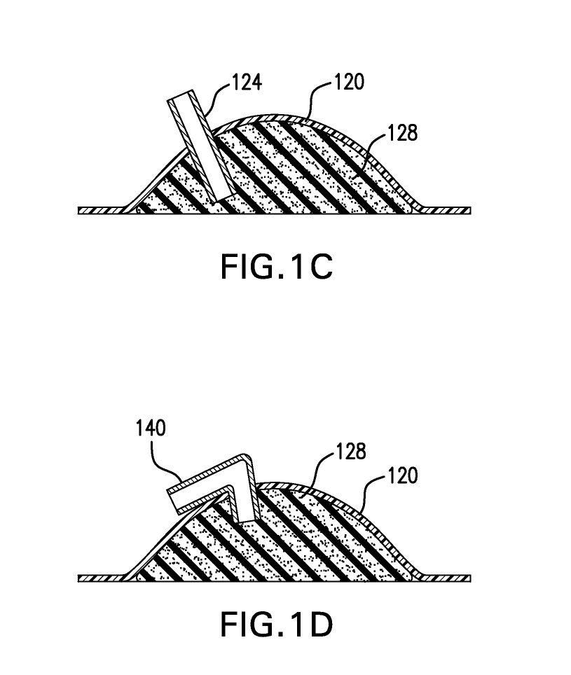

An exemplary system or apparatus 100 for dressing a wound in accordance with exemplary embodiments of the present disclosure is shown in an schematic cross-sectional illustration of FIG. 1A. The dressing system 100 can include a thin, gas-impermeable membrane 120 that can be sized, structured, configured and/or adapted to be placed over a wound 110. For example, the membrane 120 can be formed at least partially of a material such as, e.g., Tegaderm® or the like. The membrane 120 can be provided with an adhesive 122 or other sealant that can adhere the outer portions of the membrane 120 to healthy tissue 115 surrounding the wound. The adhesive 122 can be provided on the membrane 120, or alternatively or in addition, it can be provided as a tape, glue, curable material or the like that can be applied to healthy tissue 115 around the wound site 110, and the membrane then placed over the wound 110 and adhered to the surrounding tissue 115 by the adhesive 122. This exemplary configuration can provide an enclosed volume over the wound 110, e.g., a substantially sealed environment, that can facilitate maintenance of sterility and a reduced pressure therein. The membrane 120, or at least a peripheral portion thereof, can be flexible or pliable, which can facilitate shape accommodation and adherence of the membrane 120 to the tissue 115 surrounding the wound 110. In certain exemplary embodiments, a central portion of the membrane 120 can be more rigid, e.g., to form a protective “cap” over the wound 110 and/or provide a mechanically sound base for attachment or mounting of an inlet 140 and/or outlet 124 as described herein below. The membrane 120 can be provided in a size and/or shape to approximately conform to the size and/or shape of the wound 110, for example, the membrane 120 can be somewhat larger than the wound 110 such that the edges of the membrane 120 can be adhered to healthy tissue surrounding the wound 110.

The dressing system 100 can further include a resilient sponge 128 enclosed by the membrane 120 over the wound 110. The sponge 128 can be, e.g., a porous open-celled foam or similar material, such that at least a portion of the sponge 128 is permeable to fluids, e.g., liquids and/or gases. The sponge 128 can be sized and shaped appropriately to cover at least a portion of the wound 110, with a thickness that can generally be between about 1 cm and about 10 cm. The thickness of the sponge 128 can be greater or less than this size range in certain embodiments, e.g., depending on the size and location of the wound to be dressed. The peripheral or edge portion of the sponge 128 can be thinner, e.g., tapered, to better conform to the shape of the membrane 120 where it is adhered to the tissue 115 around the wound 110. A pore size of the sponge 128 can be between, e.g., about 100 μm and about 2000 μm. Other pore sizes can be used in further embodiments, and may be based on such factors as the sponge material, size of the sponge 128, etc.

The sponge 128 can maintain spacing between the membrane 120 and the wound 110, and can optionally be pre-treated or infused with any of a variety of conventional nutrients or other substances known to promote wound healing. Such substances can be provided in a timed-release form using dissolvable coatings or other time-release formulations known in the art. In certain embodiments, a lower portion of the sponge 128 configured to contact the wound surface can be provided with a material or layer to reduce or prevent adhesion of the sponge to the wound, such as a woven Teflon® mesh or the like. The sponge 128 can also facilitate removal of fluids exuded by the wound 110 away from the wound site as it heals by absorbing them and/or by allowing them to flow through the open-cell structure, or channels provided within the sponge 128 to assist in drainage of the wound 110.

The sponge 128 can also provide a mechanical force on the wound 110 when a reduced pressure or vacuum is established in the enclosed volume between the membrane 120 and the wound 110. The sponge 128 can be selected to have a desired degree of stiffness or resistance to compression/deformation. For example, the sponge 128 can be formed of or include a polymer memory foam material or the like. When a low ambient pressure is present in the volume enclosed by the membrane 120 over the wound 110, the membrane 120 may press down on the sponge 128 and in turn the sponge 128 can produce a compressive force onto the wound 110. It has been observed that such mechanical force can also promote the wound healing process, e.g., it can assist in stabilizing a tissue flap, piece of graft tissue, or the like on the wound, if present, to promote reattachment. Further, such mechanical pressure on the wound site can reduce or prevent formation of fluid pools on the wound site 110, assist in drainage of the wound 110, etc. Accordingly, the exemplary configuration shown in FIG. 1A facilitates the wound 110 to be maintained in a low-pressure environment while simultaneously applying a mechanical force onto it.

In further exemplary embodiments, the sponge 128 can be formed using two or more materials that can have different properties, or a material having a gradient in mechanical properties such as a variable stiffness. For example, certain regions of the sponge 128 can be stiffer, such as the central region, to provide a stronger resistance to deformation, whereas the peripheral portions of the sponge 128 may be softer or more resilient to better conform to the shape of the enclosed volume where the membrane 120 adheres to the surrounding tissue 115.

The dressing system 100 can include at least one outlet 124 provided through the membrane 120. The outlet 124 can optionally be provided with a flange to improve the attachment to the membrane 120 and provide a gas-tight seal between these components. Alternatively or additionally, a distal end of the outlet 124 can extend into the sponge 128 to provide mechanical support and further stabilize the outlet 124 relative to the membrane 120 as shown, e.g., in the exemplary cross-sectional side view of an exemplary embodiment illustrated in FIG. 1C. In certain exemplary embodiments, the outlet 124 can be formed as part of the membrane 120.

The outlet 124 can be provided in any location on the membrane 120 that is separate from the membrane perimeter that adheres to the tissue 115 surrounding the wound 110. A location in or near the central portion of the membrane 120 can be selected to provide a more uniform pressure distribution through the volume enclosed by the membrane 120, e.g., if the sponge 128 is or becomes resistant to flow of gases therethrough. Such resistance can arise from factors such as the permeability of the sponge material itself, partial clogging or contamination of pores or channels in the sponge 128 that can occur during the healing process, etc. In further embodiments, a plurality of outlets 124 can be provided on the membrane 120 to provide a more uniform pressure distribution through the volume enclosed by the membrane 120.

The outlet 124 can optionally be provided in an L-shape as shown, e.g., in the exemplary cross-sectional side view of an exemplary embodiment illustrated in FIG. 1D, or in another similar shape, such that the proximal end of the outlet 124 outside of the membrane 120 lies close to or along a portion of the outer membrane surface. Such exemplary configuration can provide a dressing system 100 with a lower profile over the wound 110, can help to mechanically stabilize the outlet 124, and/or can reduce the risk and effect of impacting the outlet 124 when the portion of the body containing the wound 110 moves, e.g., during physical activity of the patient.

The outlet 124 can include a one-way valve 125 that facilitates gases from the enclosed volume beneath the membrane 120 to exit therefrom, and prevents intake of gases or liquids through the outlet 124 into this enclosed volume. For example, applying an external force to the membrane 120 can deform it and compress the sponge 128 within the enclosed volume, forcing some enclosed gas out through the outlet 124 and reducing the size of the enclosed volume. The compressed sponge 128 can then exert an outward force on the membrane 120 as it tries to relax toward its larger uncompressed state, pushing the membrane 120 away from the wound 110. This can result in a reduced pressure within the enclosed volume and over the wound 110, with the one-way flow from outlet 124 allowing the membrane 120 to maintain an air-tight seal over the wound 110. In this manner, a reduced-pressure environment can be created and/or maintained over the wound 110 passively (e.g., without an external pump or power supply) by merely pressing down on the membrane 120.

In further exemplary embodiments of the present disclosure, one or more inlets 140 can be provided through the membrane 120, as shown in FIG. 1A. The inlet 140 can be configured and attached to the membrane 120 in a manner similar to the outlet 124 described above. The inlet 140 can include a valve arrangement 141 that is optionally adjustable to provide a gas-tight seal in the inlet 140 or to facilitate a controlled flow of a gas or liquid therethrough. For example, the valve arrangement 141 can be a one-way valve with optional flow adjustment or control. The valve arrangement 141 can be manually and/or automatically opened or maintained at a particular setting to facilitate a flow of ambient air or other gas into the volume enclosed by the membrane 120 when the enclosed volume is maintained under a reduced pressure. In certain embodiments, the valve arrangement 141 can be configured to provide a fixed, non-adjustable flow rate of a gas or other fluid, such as air or pure oxygen, into the reduced-pressure enclosed volume through the inlet 140.

Such air flow can provide oxygen to the wound 110 to promote healing. For example, the inlet 140 can be configured and/or controlled to admit, e.g., between about 0.1 ml/min and about 10 ml/min into the enclosed space between the membrane 120 and the wound 110. This total flow rate can be divided among a plurality of inlets 140, if present, which may provide a more uniform dispersion of oxygen or air to the wound 110. The particular inlet flow rate provided or adjusted with a particular system 100 can be selected or determined based on certain factors such as, e.g., the size of the membrane 120 (which may correspond to the size of the wound 110 being protected), the type of wound, etc. For example, the inlet flow rate can be adjusted to allow a continuous or periodic flow of oxygen and/or other substances into the enclosed volume over the wound 115, while such flow rate can be sufficiently low that a reduced pressure can be maintained within the volume. Accordingly, a periodic reduction of pressure as described herein (e.g., by compressing the sponge 128 by pressing on the membrane 120) can be sufficient to both maintain a reduced pressure over the wound and provide oxygen, moisture, and/or other substances to the wound site without the need for pressurized supply lines, powered pump arrangements, or the like.

In a still further exemplary embodiment, the sponge 128 can include one or more impermeable layers or panels 170 as shown, e.g., in the exemplary cross-sectional side view of FIG. 1B. Such panels 170 can be oriented substantially vertically to segregate the enclosed volume over the wound 110 into different compartments or areas, and/or they may be used to modify or control the compression behavior of the sponge 128. In this exemplary embodiment, the portion of the membrane 120 overlying each compartment may be provided with one or more inlets 140 and one or more outlets 124, as described herein, to facilitate flow of gases and/or liquids into and out of each compartment.

In further exemplary embodiments, the inlet 140 may be omitted from the apparatus 100, and the membrane 120 can be gas-permeable. For example, properties of the membrane 120 can be selected such that the membrane 120 facilitates a flow of air or another gas therethrough, e.g., between about 0.1 ml/min and about 10 ml/min into the enclosed volume to provide oxygen to the wound 110, while filtering out harmful substances and maintaining a sterile environment in the enclosed volume.

As described herein, the inlet 140 and/or the valve arrangement 141 can be configured to slowly or controllably introduce oxygen, e.g., as oxygen-containing air or another oxygen-containing gas, into the enclosed volume to oxygenate the wound 110 as it heals. Although such controlled “leakage” of gas into the enclosed volume may gradually increase the pressure within the enclosed volume toward the ambient pressure, the pressure can be reduced by compressing the membrane 120 again, as described above. Such compression can be performed manually, and/or it can occur “automatically” as a result of general movement of the patient. Accordingly, the flow through the inlet 140 can be adjusted to admit air or another gas into the volume while maintaining a reduced pressure in the enclosed volume over time. Such exemplary configuration can be preferable for wound healing as compared to, e.g., a completely sealed wound environment that doesn't allow introduction of oxygen. In certain exemplary embodiments, the valve arrangement 141 can be a one-way valve that prevents gases or liquids in the enclosed volume from exiting through the inlet 140, e.g., if the membrane 120 is subjected to a compressive force. In further exemplary embodiments, the inlet 140 can include a self-sealing film or the like for directly administering substances therethrough using a hypodermic needle or other delivery device.

The inlet 140 can be provided with a coupler 142 at the proximal end thereof, as shown in FIG. 1A. The coupler 142 can optionally include a filter arrangement to filter potential contaminants and prevent them from entering the enclosed volume through the inlet 140. Such filter can, for example, trap contaminants from ambient air while allowing a flow of oxygen-containing ambient air into the enclosed volume. The filter arrangement and/or the entire coupler 142 can be configured to be replaceable, e.g., to maintain permeability and filtering capability of the filter arrangement during the healing process.

In further exemplary embodiments, the coupler 142 can be configured to be attached to any of a variety of tubes, containers, reservoirs, or the like to facilitate introduction of liquid and/or gaseous substances through the inlet 140 and into the enclosed volume over the wound 110. Such exemplary substances can include, e.g., oxygen, tissue-growth promoters, antibacterial compounds, or the like. The rate at which such substances enter the enclosed volume can be facilitated by the reduced pressure within the volume and by appropriate configuration of the inlet(s) 140 and associated valve(s) 141.

The inlet 140 can also be configured to facilitate an introduction of moisture, e.g. water or an aqueous solution, into the enclosed volume over the wound 110, which can provide hydration to improve the local healing conditions. A reservoir containing water or an aqueous solution can also be affixed to the coupler 142 to provide and/or maintain hydration of the wound 110 during healing. In certain exemplary embodiments, the coupler 142 can include a water-containing material, such as a moistened absorbent filter or the like. Air or other gases that flow into the enclosed volume through the inlet 140 can be wetted by the material of the wet filter, which can both filter incoming gases and provide hydration to the wound 110. The inlet 140 can be kept open continuously or it can be opened periodically and then closed, e.g., using valve arrangement 141, to provide such gases and/or hydration to the wound site 110. A plurality of different inlets 140 can also be provided in the system 100, where each one can be configured to provide one or more of the functions described herein, e.g., to facilitate oxygen and/or water/moisture to enter the enclosed volume at a slow or controlled rate.

In an exemplary operation, the membrane 120 with the sponge 128 underneath can be applied over the wound 110, such that the membrane 128 is adhered to healthy tissue surrounding the wound 110. Such exemplary configuration can provide a sterile environment for the wound 110 to prevent contamination or exposure to contaminants, bacteria, or the like. The flexible membrane 120 can then be compressed or deformed by pushing on it, e.g., with a hand, thereby compressing the sponge 128 and expelling some of the air or gases within the enclosed space through the outlet 124. The compressed sponge 128 may then generate a restorative force that attempts to expand the internal volume enclosed by the membrane 120. This can provide a negative or lowered pressure (e.g., a pressure that is less than the ambient or atmospheric pressure) generated within the enclosed space under the membrane 120 that contains the sponge 128. In this exemplary manner, the system 100 can maintain a reduced pressure over the wound 110 while also providing some degree of mechanical force on the wound 110 by the compressed sponge 128, both of which can promote wound healing. A low flow rate of oxygen (e.g., contained in air) and/or water can be introduced into the wound site 110 through inlet 140, thereby providing additional preferable conditions to facilitate wound healing.

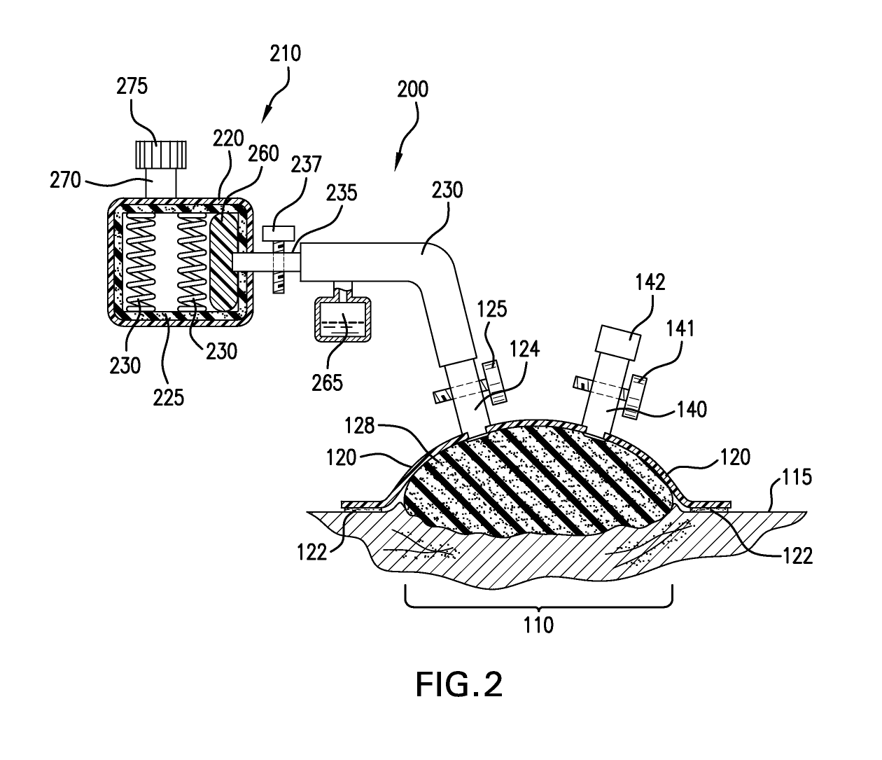

In a further exemplary embodiment according to the present disclosure, as shown in FIG. 2, a dressing system or apparatus 200 can be provided that is similar to the system 100 shown in FIG. 1A, and can further include an external vacuum arrangement 210. For example, the outlet 124 can be structured to facilitate attachment of a tube 230 to the proximal end of the outlet 124 outside of the membrane 120. The opposite end of the tube 230 can be coupled to the vacuum arrangement 210, as illustrated in FIG. 2. This exemplary configuration facilitates the wound site 110 below the membrane 120 to be in a fluid communication with the vacuum arrangement 210, thereby maintaining a reduced ambient pressure in the volume over the wound 110 enclosed by the membrane 120. The vacuum arrangement 210 can be located proximal to the membrane 120 (e.g., using a relatively short tube 230) to provide a compact system. In further exemplary embodiments, the vacuum arrangement 210 can be placed at a location remote to the membrane 120 (e.g., using a long tube 230), which can provide more options for placement and configuration of the vacuum arrangement 210 relative to the wound site and membrane 120.

The vacuum arrangement 210 can be a passive component, e.g., it can be configured and/or structured to provide a low-pressure source without requiring a battery or external source of electricity, etc. The vacuum arrangement 210 can include a housing 220, which can be air-tight or gas-impermeable and at least partially pliable or deformable, surrounding a foam 225, which can be a compressible open network that is preferably gas-permeable. The foam 225 can be sufficiently rigid to exert a restoring force when the housing 220 surrounding the foam 225 is compressed, e.g., to return the housing 220 to a relaxed size or expanded volume. For example, the foam 225 can include a polymer memory foam material or the like. In some exemplary embodiments, the foam 225 can be a closed-cell material, with channels or passages provided therethrough to facilitate flow of gases, while the foam 225 can provide a restoring force when compressed to expand the housing 220 and withdraw gases from the enclosed volume over the wound 110 to reduce the pressure, as described herein. In further exemplary embodiments, the foam 225 can be very pliable and easily compressed (e.g., very soft), and one or more optional spring-like structures 230 can be provided within the housing 220 that are configured to restore the housing 220 towards an expanded state when it is compressed. Such spring-like structures 230 can include, e.g., springs, a rubber balls or the like, a resilient framework provided within or formed as part of the housing 220, etc. In certain exemplary embodiments, the vacuum arrangement 210 can include the housing 220 and the spring-like structures 230 with no internal foam 225.

The vacuum arrangement 210 can include one or more intake ports 235, e.g., mounted on or formed as part of the housing 220. The intake port 235 can provide a fluid path or communication between the interior and exterior of the housing 220. For example, it can include a valve arrangement 237, e.g., a one-way valve, configured to allow gases and/or liquids to enter the interior volume of the housing 220 from the proximal end of the intake port 235 when opened, and prevent gases or liquids from entering or leaving the interior of the housing 220 via the intake port 235 when the intake valve arrangement 237 is closed. The intake port 235 can be configured and/or structured to couple or attach to the tube 230 or other conduit or fitting, and thereby provide a source of low pressure when coupled to an external conduit or enclosure such as, e.g., the enclosed volume between the membrane 120 and the wound 110.

A trap 260 can optionally be provided within a portion of the intake port 235 and/or within the housing 220 proximal to the intake port 235. Such exemplary trap 260 can retain moisture, liquids, particles, impurities, or the like that may enter the intake port 235 during use. The trap 260, if present, can prevent clogging and/or contamination of the foam 225. The trap 260 can be formed, at least in part, from one or more materials such as, e.g., a paper filter element, a woven material, a filter screen, an open-cell sponge or scaffold material, an absorbent material, or a combination of such materials.

In certain exemplary embodiments, a conventional superabsorbent polymer (SAP) can be used in the trap 260 to absorb fluids. Superabsorbent polymers can be made, e.g., from a polymerization reaction of acrylic acid blended with sodium hydroxide in the presence of an initiator to form a poly-acrylic acid sodium salt. Other types of SAPS can also be used in embodiments of the present disclosure. For example, SAPs are commonly used in baby diapers and similar products. In another embodiment, a portion of the sponge 128 can be formed using an SAP, e.g., the portion of the sponge 128 proximal to the wound 128 (or the periphery of the wound 110) to facilitate drainage of the wound 110.

In further exemplary embodiments, an optional fluid trap 265 can be provided as part of or coupled to the tube 230, instead of or in addition to the trap 260. Such exemplary fluid trap 265 can have the form of a container or vessel to collect entrained fluids or particles as flow occurs through the tube 230, as shown in FIG. 2, and it can be configured to be removable or replaceable, e.g., if it fills up or becomes clogged. In further exemplary embodiments, the trap 265 can be provided as an inline cartridge or the like, e.g., in a configuration similar to that of a conventional gas line filter in an automobile, such that gases and fluids passing through the tube 230 can flow through the fluid trap 265. In this exemplary configuration, the fluid trap 265 can be disposable and replaceable, e.g., by removing it from the tube 130 and attaching a new one in its place as needed.

The trap 260 and/or the fluid trap 265 can include, e.g., a hydrophilic or absorbent material, such as an SAP or the like, that can facilitate absorption of fluids drained from the wound 110 that travel through the tube 230. For example, the tube 230 can be configured and/or structured to facilitate flow of such drainage fluids from the wound site 110 through the tube 230 and into the trap 260 and/or fluid trap 265, if present. Such flow can be facilitated by gravity, e.g., if the tube 230 and or traps 260, 265 are configured, oriented and/or mounted appropriately. For example, the system 200 can be configured such that the trap 260 and/or fluid trap 265, if present, are located below the outlet 124 at least occasionally or periodically (e.g., when the patient body associated with the wound 110 is lying down, sitting, or standing up), such that fluids within the enclosed space under the membrane 120 can exit the outlet 124 and be retained by the trap 260 and/or fluid trap 265.

In still further exemplary embodiments, the system 100 shown in FIG. 1A can be provided with a fluid trap 265 coupled or connected directly to the outlet 124, e.g., where the fluid trap 265 can be configured, structured and/or arranged to facilitate a drainage of fluids from the wound site 110 as described herein.

In yet further exemplary embodiments of the present disclosure, the system 100, 200 shown in FIGS. 1A and 2, respectively, can be provided with an absorbent material (e.g., a superabsorbent polymer or the like) that can be provided below the membrane 120 and proximal to the wound 110, e.g., near the periphery of the membrane 120. This absorbent material can absorb fluids produced at the wound site, e.g., to facilitate drainage of the wound 110 as it heals, and can be replaced occasionally or retained for the duration of the healing process until the dressing system 100, 200 is removed.

The vacuum arrangement 210 can further be provided with one or more exhaust ports 270. The exhaust port 270 can include a one-way valve that allows gases to exit from the interior of the housing 220, but prevent gases or other substances from entering the interior of the housing 220 through the exhaust port 270. An exhaust trap 235 can optionally be provided at the distal end of the exhaust port 270, e.g., to prevent liquids, particles, or other substances from being released from the interior of the housing 220 to the surroundings. For example, the exhaust trap 235 can include an absorbent material, a filter, or a combination of these elements.

In an exemplary operation, the housing 220 of the vacuum arrangement 210 can be compressed or deformed, facilitating enclosed air or gases to exit through the exhaust port 270. Gases or fluids can be prevented from exiting the housing 220 through the intake port 235 during this procedure by the one-way valve arrangement 237. The compressed foam 225 and/or deformed housing 220 (and/or spring-like structures 230 inside the housing 220, if present) can then generate a restorative force that attempts to expand the internal volume of the housing 220. This can provide a negative pressure source generated within the tube 230 via the intake port 235. The amount of such restorative force can vary with several factors, including but not limited to the size and shape of the housing 220, the material(s) of the housing 220 and foam 225, the geometry and material of any internal spring-like structures 230 (if present), etc.

If gases or liquids enter the vacuum arrangement 210 over time, e.g., if drawn in from the enclosed volume beneath the membrane 120 via tube 230 and through the intake port 235, the lowered pressure provided within this enclosed volume over the wound 110 can be reduced over time as the housing 220 expands towards its relaxed state and the restorative forces diminish. The low-pressure source can be “recharged” by deforming the housing 220 again, expelling some of the gases contained therein through exhaust 270, as described above.

The vacuum arrangement 210 can be provided in various shapes and sizes. Selection of a particular shape and/or size can be based on factors such as the range of lowered pressures desired at the intake port 235 and/or within the enclosed volume over the wound 110, the “longevity” of the low-pressure vacuum arrangement 210 between recharges as gases enter the intake port 235 over time, where the vacuum arrangement 210 can be used, etc. For example, the vacuum arrangement 210 can be in a shape of a bulging disc or oval, a cylinder, a bellows, an elongated tube, or the like. Accordingly, specific properties of a particular vacuum arrangement 210 can thus be selected without extensive experimentation based on the flow or “leakage” rate of the enclosed volume connected to the vacuum arrangement 210 via tube 230, the desired reduced pressure, etc.

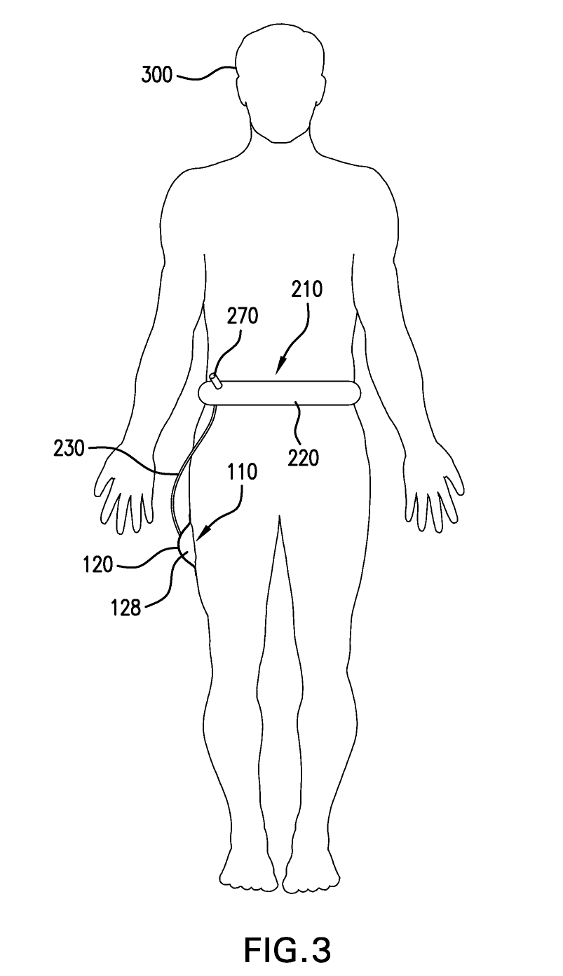

For example, when provided as part of the exemplary dressing arrangement 200, the vacuum arrangement 210 can be shaped and configured to be attached or adhered to a body part proximal to the wound 110, placed in a clothing pocket etc. The vacuum arrangement 210 can be configured to periodically undergo slight compressions during normal bodily activity to maintain the housing 220 in a compressed state, or it can be configured to be manually deformed periodically, e.g., once or a few times per day, once per week, etc., or both. In certain embodiments, the vacuum arrangement 210 can be provided on the chest or stomach area, under an arm, behind a knee, etc., and can be affixed to the body using a strap or band, an adhesive, hook-and-loop closures, or the like. Such a configuration allows the vacuum arrangement 210 to be deformed by normal body motion such as breathing, arm movement, walking, etc. Activation of the vacuum arrangement 210 by such bodily movements can be achieved without conscious effort by the patient, and thus can be considered to be passive or “automatic” with no external power source or directed manipulation needed to maintain low pressure over the wound site. For example, the exemplary configuration of the vacuum arrangement 210 shown in FIG. 3 includes a housing 220 that is wrapped at least partially around the chest of a subject 300. Such configuration can provide compression of the foam 225 (not shown) within the housing 220 based on the natural expansion and contraction of the chest area when breathing, which can maintain a low pressure within the housing 220 and tube 230 over time without any external power source or directed action by the subject 300. Such low pressure can be maintained over a wound area 110 that is located under the dressing 120, via tube 230, even when a flow of gas through inlet 140 into the enclosed area over the wound 110 occurs, e.g., to keep the wound 110 oxygenated and/or hydrated.

In further exemplary embodiments, the various valves and ports (e.g. intake port 235, outlet 124, etc.) can be configured such that periodic deformation of the vacuum arrangement 210 can vary the pressure level within the volume enclosed by the membrane 120, e.g., from a reduced pressure to a less-reduced pressure, or from a reduced pressure to a pressure greater than ambient pressure. Such variations in pressure over time can also promote wound healing.

In yet further exemplary embodiments, the vacuum source 210 used with the wound dressing system 200 can be a conventional low-pressure or vacuum source, e.g., a mechanical pump or bellows, a hydraulic pump, etc., although such vacuum sources may be bulkier, more costly, and/or less convenient than the embodiment of a vacuum arrangement 210 shown in FIG. 2 and described above. However, such conventional vacuum sources, in combination with the other features described herein, can provide a low-pressure environment for the wound 110 while also keeping the wound site sterile, hydrated, oxygenated, etc.

In another exemplary embodiment of the present disclosure, one or more sensors (not shown) can be provided in the system 100, 200. For example, a pressure sensor can be provided at one or more locations within the system to detect, e.g., the pressure within the enclosed volume over the wound 110 or within the vacuum arrangement 210. Such pressure sensors can indicate blockage or obstruction of a component, e.g. the fluid trap 265, an inlet 140 or outlet 124, etc. A pH sensor can also be provided in certain embodiments to monitor the conditions close to the healing wound 110. The pH sensor can be provided, for example, as a conventional color-changing strip, area, or coating located proximal to the wound 110.

The presence of oxygen at a wound site can promote healing. As described above, oxygen can be introduced to the wound site by allowing a flow of ambient air or other oxygen-containing gas to enter the inlet 140, either continuously by opening the valve arrangement 141 such that air is drawn into the enclosed volume by the pressure difference at a desired or particular volumetric flow rate, or by periodically opening and closing the valve arrangement 141 of the inlet 140. Alternatively or in addition, a source of oxygen can be connected to the coupler 142, and the valve arrangement 141 opened to introduce oxygen from the controlled oxygen source into the enclosed volume over the wound. An oxygen sensor can be provided to detect oxygen level within the enclosed volume over the wound 110, or optionally to provide a signal when the oxygen level drops below a particular value. An oxygen sensor can have a form of, e.g., a color-changing material or coating provided within the enclosed volume (e.g., on the inner surface of the membrane 120), which can indicate a presence or lack of sufficient oxygen at the wound site based on the visible color of the sensor. Other oxygen-level indicators known in the art can also be used. The oxygen sensor can, e.g., provide notification when oxygen levels drop below a particular level and indicate that the inlet 140 should be manually open or the inlet flow rate should be increased. In further embodiments the oxygen sensor can be used, for example, to automatically open a valve arrangement in an inlet 140 and admit more air or oxygen into the enclosed volume, although such sensing and control arrangements may increase the cost and/or complexity of the system 100, 200.

In a further exemplary embodiment, an oxygen-forming compound can be provided within the enclosed volume, e.g., distributed within or coating a portion of the sponge 128 and/or membrane 120. For example, calcium peroxide (CaO₂) can be provided within the enclosed volume. This compound slowly decomposes when contacted by water to form calcium hydroxide and oxygen. Fluids exuded by the wound and/or water introduced through the inlet 140 can be used to activate the reaction and generate small amounts of oxygen within the enclosed volume over time. Other sources of oxygen known in the art can be used in a similar manner with the wound dressing system 100, 200 in further embodiments of the present disclosure.

In still further exemplary embodiments, at least a portion of the membrane 120 and/or sponge 128 can be formed of particular materials that facilitate transmission of light having certain wavelengths. This optical transmissivity of portions of the dressing system 100 can facilitate treatment of the wound 110 by irradiating it with light having particular wavelengths, intensity, and duration. Such light can be provided, e.g., by any one or more of a variety of light-producing devices known in the art. Certain such optical therapies are known in the art, and a selection of materials with suitable optical properties can be based on the particular type of optical energy to be used.

In further exemplary embodiments, one or more LEDs or other light-emitting arrangements (not shown) can also be provided on or over, or affixed to, the membrane 120, or provided within the enclosed volume over the wound 110 (e.g., placed on or in a portion of the sponge 128). Such light-emitting arrangements can be battery-powered for portability, and can be configured to emit light at one or more wavelengths known in the art to enhance the healing process. These light-emitting arrangements can also be used to activate any photosensitive substances that may be introduced into the system 110 to provide a phototherapy treatment of the wound 110.

Accordingly, certain exemplary embodiments of the present disclosure can provide wound dressing system and method that provides a number of beneficial factors and conditions to promote wound healing. The exemplary system can be passive in nature, e.g., requiring no electrical power source or connection, and can maintain a reduced pressure over the wound while maintaining a mechanical force on the wounded tissue. Powered vacuum arrangements, sensors, controlled valves, displays, etc. can also be provided with the system in further embodiments. The system 100, 200 can facilitate an introduction of various healing-promoting substances to the wound, including oxygen and moisture, while maintaining a sterile environment. Although particular embodiments of the present disclosure are illustrated in FIGS. 1A and 2, other exemplary configurations that embody the exemplary principles and functions herein can be used in further embodiments and are within the scope of the present disclosure. For example certain exemplary components of the exemplary wound dressing system 100, 200 can have different sizes, shapes, and/or numbers than those illustrated herein (e.g., there can be more than one inlet, outlet, intake port, exhaust port, membrane, etc.).

The foregoing merely illustrates the principles of the present disclosure. Various modifications and alterations to the described embodiments will be apparent to those skilled in the art in view of the teachings herein. It will thus be appreciated that those skilled in the art will be able to devise numerous techniques which, although not explicitly described herein, embody the principles of the present disclosure and are thus within the spirit and scope of the present disclosure. All patents and publications cited herein are incorporated herein by reference in their entireties.

1. An apparatus for improving healing behavior of a biological wound, comprising:
a membrane configured to form an enclosed volume over the wound;
an open-cell sponge provided within the enclosed volume;
at least one inlet arrangement configured to provide at least one of oxygen and moisture to the wound;
an outlet arrangement configured to facilitate removal of at least one of a gas or a liquid from the enclosed volume; and
a vacuum arrangement configured to provide a reduced pressure within the enclosed volume,
wherein the sponge is configured to provide a force on the wound when a reduced pressure is present in the enclosed volume.

2. The apparatus of claim 1, wherein the at least one inlet arrangement comprises an inlet valve arrangement configured to control a flow of a gas into the enclosed volume when the reduced pressure is present in the enclosed volume.

3. The apparatus of claim 2, wherein a proximal end of the at least one inlet arrangement extends into the sponge within the enclosed volume.

4. The apparatus of claim 2, wherein the at least one inlet arrangement further comprises a filter arrangement structured to filter the gas flowing into the enclosed volume.

5. The apparatus of claim 4, wherein the filter arrangement comprises a wetted material structured to add moisture to the gas flowing into the enclosed volume through the filter arrangement.

6. The apparatus of claim 1, wherein the outlet arrangement comprises an outlet valve arrangement configured to prevent a flow of a gas into the enclosed volume when the reduced pressure is present in the enclosed volume.

7. The apparatus of claim 1, further comprising a quantity of calcium peroxide provided within the enclosed volume, whereby the calcium peroxide is provided in a form to react with moisture within the enclosed volume to release oxygen therein.

8. The apparatus of claim 1, further comprising at least one light-emitting arrangement configured to emit light onto the wound when the apparatus is placed over the wound.

9. The apparatus of claim 1, further comprising at least one of an oxygen sensor, a pressure sensor, or a pH sensor.

10. The apparatus of claim 1, wherein the vacuum arrangement comprises the membrane, the sponge, and the outlet arrangement, and wherein the vacuum arrangement is structured to remove a volume of gas from the enclosed volume through the outlet arrangement when the sponge is compressed.

11. The apparatus of claim 1, wherein the vacuum arrangement comprises:
a gas-impermeable housing that is at least partially deformable;
a resilient structure provided within the housing;
a port comprising an opening through the housing;
an exhaust arrangement comprising a valve arrangement and a further opening through the housing; and
a tube connecting the port to the outlet arrangement,
and wherein the vacuum arrangement is structured to remove a gas from the enclosed volume through the outlet arrangement when the housing is compressed.

12. (canceled)

13. The apparatus of claim 11, wherein the resilient structure comprises (i) at least one of an open-cell foam provided within the housing or (ii) a spring-like structure that is at least one of provided within the housing or formed as part of the housing.

14. The apparatus of claim 11, wherein the vacuum arrangement is configured to expel gas through the exhaust arrangement and provide a reduced pressure within the enclosed volume when the housing is compressed.

15. The apparatus of claim 11, further comprising a fluid trap coupled to at least one of the outlet arrangement, the tube, and the housing, wherein the fluid trap is structured to remove moisture from a gas that flows from the enclosed volume into the vacuum arrangement.

16. (canceled)

17. A method for providing a dressing for a biological wound, comprising:
adhering a membrane to healthy tissue surrounding the wound to form an enclosed volume over the wound;
providing oxygen and moisture to the wound;
providing a reduced pressure within the enclosed volume; and
providing an open-cell sponge arrangement within the enclosed volume that is configured to provide a mechanical force on the wound when the reduced pressure is present within the enclosed volume.

18. The method of claim 17, wherein at least one of the oxygen and the moisture is provided in a gas that is directed to flow into the enclosed volume through a first opening provided in the membrane.

19. The method of claim 18, wherein the reduced pressure is provided by compressing the membrane to force air to flow out of the enclosed volume through a second opening provided in the membrane.

20. The method of claim 18, wherein the reduced pressure is provided by compressing an external resilient housing to direct air to flow out of the enclosed volume through a second opening provided in the membrane and into the housing via a tube connecting the second opening and a further opening in the housing.

21. The method of claim 17, wherein the oxygen is provided by a chemical reaction within the enclosed volume.

22. The method of claim 21, wherein the chemical reaction comprises an interaction between water and calcium peroxide provided within the enclosed volume.
